(19) United States
(12) Patent Application Publication
(10) Pub. No.: US 2025/0261918 A1
(43) Pub. Date: Aug. 21, 2025

(54) X-RAY IMAGING SYSTEM AND X-RAY IMAGE DISPLAY METHOD

(71) Applicant: SHIMADZU CORPORATION, Kyoto-shi (JP)

(72) Inventor: Takahiro KAWAMURA, Kyoto-shi (JP)

(21) Appl. No.: 19/031,554

(22) Filed: Jan. 18, 2025

(30) Foreign Application Priority Data
Feb. 21, 2024 (JP) 2024-024949

(51) Int. Cl. A61B 6/46; A61B 5/103; A61B 6/00; A61B 6/50

(52) U.S. Cl. CPC A61B 6/463; A61B 5/1036; A61B 6/505; A61B 6/5247

(57) ABSTRACT

An X-ray imaging system according to this invention includes an X-ray irradiator for irradiating a subject who is in loaded action in which a load is applied to legs of the subject with X-rays; an X-ray detector; a load information acquirer for acquiring load information to identify a state of the load applied to the leg or the legs in the loaded action of the subject separately from the detection of the X-rays; a display; and a controller. The controller executes control for identifiably displaying at least an X-ray image that corresponds to a maximum load timing in which a largest load is applied to one of legs of the subject in loaded action on the display based on the load information.

### CROSS-REFERENCE TO RELATED APPLICATIONS

The related application number JP2024-024949, X-ray imaging system and X-ray image display method, Feb. 21, 2024, KAWAMURA Takahiro, upon which this patent application is based are hereby incorporated by reference.

### BACKGROUND OF THE INVENTION

### Field of the Invention

The present invention relates to an X-ray imaging system and an X-ray image display method.

### Description of the Background Art

X-ray imaging systems including optical image capturers are known in the art. Such a system is disclosed in Japanese Patent Laid-Open Publication No. JP 2021-058570, for example.

The above Japanese Patent Laid-Open Publication No. JP 2021-058570 discloses a radiography system for capturing a radiographic image using X-rays. In this radiography system, video is captured while a joint is moved under a load to observe movements of the joint and bones in walking or the like.

Here, in a case in which X-ray images of a subject during loaded action such as walking action in which a load is applied are captured as in the radiography system disclosed in the above Japanese Patent Laid-Open Publication No. JP 2021-058570, a physician may make diagnosis by observing a state of the bones or the joint in the timing in which the largest load is applied to one of the subject's legs. However, in the case in which the X-ray images are captured while the subject is in loaded action, it is difficult for the physician to identify the maximum load timing in which the largest load is applied to one of the legs only by seeing the X-ray images. For this reason, it is desired to realize easy identification of an X-ray image that corresponds to the maximum load timing in which the largest load is applied to one of the legs.

### SUMMARY OF THE INVENTION

The present invention is intended to solve the above problem, and one object of the present invention is to provide an X-ray imaging system and an X-ray image display method capable of realizing easy identification of an X-ray image that corresponds to a maximum load timing in which the largest load is applied to one of legs.

An X-ray imaging system according to a first aspect of the present invention includes an X-ray irradiator for irradiating a subject who is in loaded action in which a load is applied to legs of the subject with X-rays; an X-ray detector for detecting the X-rays with which the subject is irradiated by the X-ray irradiator and that pass through the subject; a load information acquirer for acquiring load information to identify a state of the load applied to the leg or the legs in the loaded action of the subject separately from the detection of the X-rays using the X-ray detector; a display for displaying an X-ray image(s) generated based on the detection of the X rays using the X-ray detector; and a controller for executing control for identifiably displaying at least the X-ray image that corresponds to a maximum load timing in which a largest load is applied to one of the legs in the loaded action of the subject on the display based on the load information acquired by the load information acquirer.

An X-ray image display method according to a second aspect of the present invention includes a step of irradiating a subject who is in loaded action in which a load is applied to legs of the subject with X-rays; a step of detecting the X-rays that pass through the subject; a step of acquiring load information to identify a state of the load applied to the leg or the legs in the loaded action of the subject separately from the detection of the X-rays; and a step of displaying an X-ray image(s) generated based on the detection of the X-rays, wherein the step of displaying the X-ray image(s) includes a step of identifiably displaying at least the X-ray image that corresponds to a maximum load timing in which a largest load is applied to one of the legs in the loaded action of the subject based on the load information acquired.

In the aforementioned X-ray imaging system according to the first aspect and the aforementioned X-ray image display method according to the second aspect, as described above, at least an X-ray image that corresponds to a maximum load timing in which a largest load is applied to one of legs of a subject in loaded action based on load information acquired is identifiably displayed. Accordingly, since the X-ray image that corresponds to the maximum load timing is identifiably displayed, easy identification of the X-ray image that corresponds to the maximum load timing in which the largest load is applied to one of the legs can be realized by allowing visual recognition of the X-ray image displayed that corresponds to the maximum load timing.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic view showing an entire configuration of an X-ray imaging system according to a first embodiment.

FIG. 2 is a block diagram showing the overall configuration of the X-ray imaging system according to the first embodiment.

FIG. 3 is a schematic view showing an exemplary display screen of a display displaying an X-ray image and an optical image.

FIG. 4 is a view showing an exemplary display screen displaying a plurality of X-ray images and a plurality of optical images in alignment with each other.

FIG. 5 is a flowchart illustrating control processing in an X-ray image display method using the X-ray imaging system according to the first embodiment.

FIG. 6 is a block diagram showing an overall configuration of an X-ray imaging system according to a second embodiment.

FIG. 7 is a flowchart illustrating detection of a position of a leg in a height direction according to the second embodiment.

FIG. 8 is a chart schematically showing exemplary periodic change of difference between a position of a toe of a right leg and a position of a toe of a left leg in an upward\downward direction of an optical image 80.

FIG. 9 is a schematic chart illustrating setting of X-ray irradiation timing.

FIG. 10 is a view showing an exemplary display screen of the display displaying an X-ray image corresponding to a maximum load timing.

FIG. 11 is a flowchart illustrating control processing of an X-ray image display method using the X-ray imaging system according to the second embodiment.

FIG. 12 is a schematic view showing an entire configuration of an X-ray imaging system according to a third embodiment.

FIG. 13 is a schematic diagram illustrating setting of X-ray irradiation timing based on detection results of a pressure sensor.

### DESCRIPTION OF THE PREFERRED EMBODIMENT

Embodiments embodying the present invention will be described with reference to the drawings.

### First Embodiment

### (Configuration of X-Ray Imaging System)

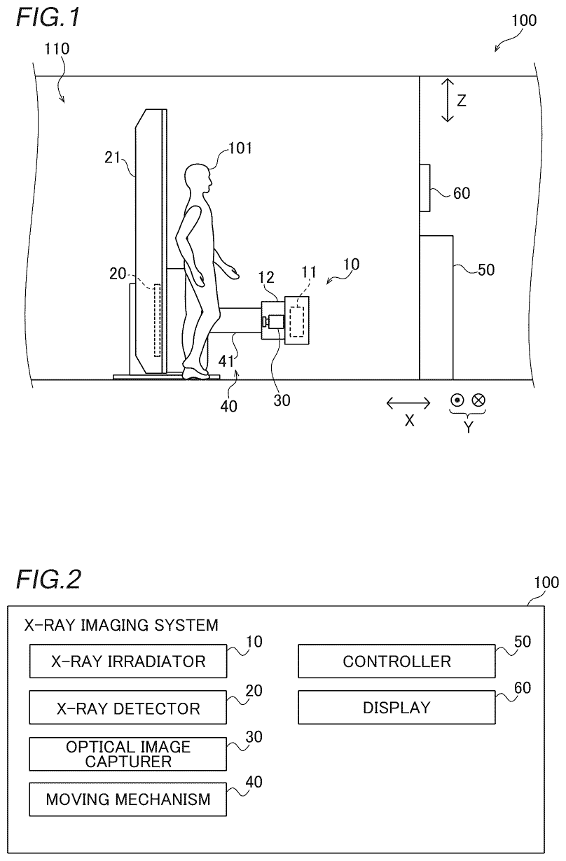

The following description describes a configuration of an X-ray imaging system 100 according to a first embodiment of the present invention with reference to FIGS. 1 to 4.

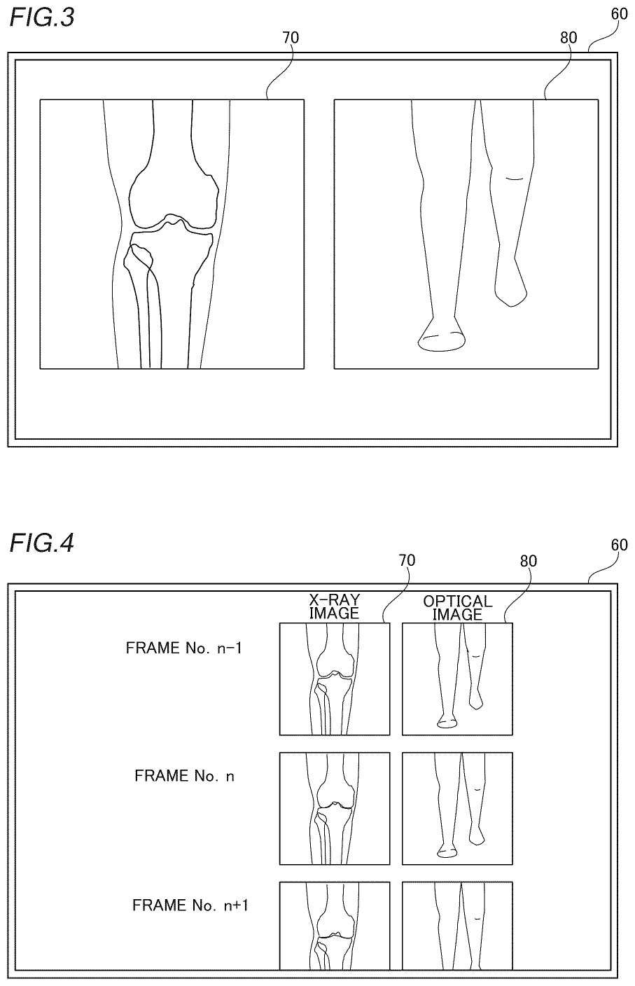

As shown in FIG. 1, the X-ray imaging system 100 is a system including a medical X-ray imaging apparatus (so-called X-ray fluoroscopy system) for capturing X-ray images 70 (see FIG. 3) as video by successively irradiating a subject 101 with X-rays, and is configured to capture the X-ray images of the subject as an imaging target. For example, before and after artificial joint replacement surgery in a knee joint or the like, X-ray images of the subject 101 are captured during walking action as loaded action in which a load is applied to the subject's legs. In this capture of the images, since bones overlap each other in the knee joint in the X-ray image 70 of the subject's knee being bent, visibility decreases in the X-ray image 70. For this reason, a physician will use the X-ray image 70 in a timing in which the knee is extended and receives a load to make diagnosis. In the first embodiment, the X-ray imaging system 100 provides assistance for identification of the X-ray image 70 that corresponds to a maximum load timing in which a largest load is applied to one of the legs in the loaded action from the plurality of X-ray images 70, which are successively captured and each of which is captured as one frame of video. Here, in a case in which the loaded action is walking action, the subject can walk in place or walk on a treadmill.

As shown in FIG. 2, the X-ray imaging system 100 includes an X-ray irradiator 10, an X-ray detector 20, an optical image capturer 30, a moving mechanism 40, a controller 50, and a display 60. In the X-ray imaging system 100, the X-rays with which the subject is irradiated by the X-ray irradiator 10 are detected by the X-ray detector 20 to capture the X-ray images of the subject 101. In the X-ray imaging system 100, optical images 80 (see FIG. 3) of the exterior of the subject 101 are captured by the optical image capturer 30. As shown in FIG. 1, in the X-ray imaging system 100, for example, the X-ray irradiator 10, the X-ray detector 20, the optical image capturer 30, and the moving mechanism 40 are installed in an imaging room 110, and the controller 50 and the display 60 are installed outside the imaging room 110. Here, the controller 50 and the display 60 may be installed inside the imaging room 110. Also, the optical image capturer 30 is an example of a “load information acquirer” in the claims. Also, the optical image 80 is an example of “load information” in the claims.

The X-ray irradiator 10 irradiates the subject 101 during the loaded action with the X-rays. The X-ray irradiator 10 includes an X-ray tube 11 and a collimator 12. The X-ray irradiator 10 is configured to irradiate the subject 101 with the X-rays from the X-ray tube 11. The X-ray tube 11 is configured to emit X-rays for irradiation when a predetermined voltage is applied. The collimator 12 includes a plurality of position-adjustable shielding plates (collimator leaves). The collimator 12 is configured to specify (adjust) an irradiation field of the X-rays emitted from the X-ray tube 11 for irradiation by shielding a part of the X-rays from the X-ray tube 11. The collimator 12 is arranged in proximity to the X-ray tube 11 in an X-ray irradiation direction of the X-ray tube 11.

The X-ray detector 20 is configured to detect the X-rays with which the subject is irradiated by the X-ray irradiator 10 and that pass through the subject 101. For example, the X-ray detector 20 is movably arranged in a bed 21 of the X-ray fluoroscopy system. The X-ray detector 20 includes a flat panel detector (FPD). The X-ray detector 20 outputs signals based on the X-rays detected to the controller 50.

The X-ray irradiator 10 is movably held in the imaging room 110 by the moving mechanism 40. Here, a vertical (upward/downward) direction is defined as a Z direction, and two directions perpendicular to each other in horizontal directions are defined as X and Y directions. The moving mechanism 40 is configured to movably hold the X-ray irradiator 10. The moving mechanism 40 includes a support 41 supporting the X-ray irradiator 10. The moving mechanism 40 moves the X-ray irradiator 10 with the X-ray irradiator facing the bed 21 in which the X-ray detector 20 is arranged. The moving mechanism 40 swings the bed 21 and the support 41 together with each other to an erected/laid orientation, for example. Also, the support 41 changes a distance between the X-ray irradiator 10 and the bed 21 in which the X-ray detector 20 is arranged. In the first embodiment, the bed 21 is orientated in the erected orientation to capture images of the subject 101 in the walking action from an X-direction side, which is a horizontal-direction side. In other words, the X-ray irradiator 10 and the X-ray detector 20 are arranged with facing each other in the horizontal direction (X direction). The moving mechanism 40 includes an electric motor as a drive source, for example.

The optical image capturer 30 captures the optical images 80 (see FIG. 3) of the subject 101 during the loaded action. The optical image capturer 30 acquires the optical images 80 as load information to identify a state of the load applied to the leg in the loaded action of the subject 101 separately from the detection of the X-rays using the X-ray detector 20. In the first embodiment, the optical image capturer 30 is arranged on an exterior surface of the collimator 12. The optical image capturer 30 is arranged with facing an X-ray irradiation direction side of the X-ray irradiator 10. The optical image capturer 30 captures the optical images 80 of the subject 101 from an X-ray irradiator 10 side when the X-ray irradiator 10 faces toward the subject 101 and the X-ray detector 20. The optical image capturer 30 is an optical camera, for example. The optical image capturer 30 includes an imaging element such as a CCD (Charge Coupled Device) image sensor or CMOS (Complementary Metal Oxide Semiconductor) image sensor, for example. The optical image capturer 30 transmits and receives signals to and from the controller 50 through a wireless or wired connection. The optical image capturer 30 outputs signals detected by the image sensor to the controller 50. The optical image capturer 30 is configured to be directed by the controller 50 to capture the optical images 80 in timings that are synchronized with detection of the X-rays using the X-ray detector 20 separately from the detection of the X-rays using the X-ray detector 20.

The controller 50 performs control of X-ray imaging using the X-ray irradiator 10 and the X-ray detector 20, and control relating to the capture of the optical images 80 using the optical image capturer 30. Specifically, the controller 50 includes a CPU (Central Processing Unit). The controller 50 controls the X-ray imaging and the capture of the optical images 80 based on parameters and various programs, which are previously set and stored in a storage. Also, the controller 50 controls displaying of the X-ray and optical images 70 and 80 on the display 60.

As shown in FIG. 3, the display 60 displays the X-ray image 70 that is generated by the controller 50 based on the detection of the X-rays using the X-ray detector 20. Also, the display 60 displays the optical image 80 that is generated by the controller 50 based on the image capture using the optical image capturer 30. The display 60 includes a liquid crystal display, for example.

### (Control of Displaying of X-Ray and Optical Images)

As shown in FIG. 4, in the first embodiment, the controller 50 is configured to control the display 60 to identifiably display the X-ray image 70 that corresponds to the maximum load timing based on the optical images 80 captured (acquired) by the optical image capturer 30. In the first embodiment, the X-ray images 70 of a knee joint in a right leg as the one of the legs, and the optical images 80 including parts from knee joints to toes are acquired and displayed on the display 60.

Specifically, in the first embodiment, the controller 50 generates a plurality of X-ray images 70 by successively irradiating the subject 101 in the loaded action with the X-rays. The controller 50 directs the X-ray irradiator 10 to successively irradiate the subject with the X-rays at a predetermined irradiation rate, which is previously set, and acquires detection signals of the X-rays with which the subject is irradiated at the predetermined irradiation rate and that are detected by the X-ray detector from the X-ray detector 20. The predetermined irradiation rate is ten exposures per second, for example. The controller 50 generates the plurality of X-ray images 70 based on the detection signals of the X-rays from the X-ray detector 20. Each of the plurality of X-ray images 70 is an image of each frame of video, for example.

Also, the controller 50 acquires the optical images 80 as video captured by the optical image capturer 30 at a frame rate not smaller than the X-ray irradiation rate. In the first embodiment, the controller 50 sets an imaging rate (period) of the optical image capturer 30 at the same frame rate as the X-ray irradiation rate. The controller 50 captures the optical images 80 as video at the same frame rate and with the same timing as the X-ray irradiation rate at which the plurality of X-ray images 70 are acquired. The optical images 80 as video include a plurality of optical images 80 as still images. The controller 50 generates the plurality of optical images 80, which are images of the exterior of the subject 101 successively captured, based on signals from the optical image capturer 30. The controller 50 controls operations of the X-ray irradiator 10 and the X-ray detector 20, and an operation of the optical image capturer 30 to synchronize these operations with each other so that a timing in which each of the plurality of X-ray images 70 is captured as one frame of video is synchronized with a timing in which each of the plurality of optical images 80 is captured as one frame of video.

In the first embodiment, to display the X-ray image 70 that corresponds to the maximum load timing, the controller 50 captures the X-ray images 70 and the optical images 80 as video during a period of one cycle of the walking action. For example, the controller 50 captures the X-ray images 70 and the optical images 80 as video during a period of time of a series of motions from a state in which a foot of the one of the legs leaves the ground through a state in which a foot of another of the legs leaves the ground and to a state in which the foot of the one of the legs leaves the ground again in the walking action of the subject 101 as the period of one cycle of the walking action. Accordingly, the plurality of X-ray images 70 and the plurality of optical images 80 generated by the controller 50 include one X-ray image 70 and one optical image 80 that correspond to the maximum load timing in which the largest load is applied to the one of the legs in the walking action.

As shown in FIG. 4, in the first embodiment, the controller 50 directs the display 60 to display one of the plurality of X-ray images 70 generated and one of the plurality of optical images 80 generated in alignment with each other for each frame. In other words, since the plurality of X-ray images 70 include the X-ray image 70 that corresponds to the maximum load timing, and the plurality of optical images 80 includes the optical image 80 that corresponds to the maximum load timing, in the first embodiment, the controller 50 displays the X-ray image 70 that corresponds to the maximum load timing in association with the optical image 80 that corresponds to the maximum load timing on the display 60 by displaying the X-ray image 70 that corresponds to the maximum load timing and the optical image 80 that corresponds to the maximum load timing in alignment with each other on the display 60. The physician determines which of the plurality of optical images 80 is the optical image 80 that corresponds to the maximum load timing in the plurality of optical images 80 based on the exterior of the leg by visually recognizing the plurality of optical images 80 displayed on the display 60 frame by frame. For example, the physician determines the optical image 80 that corresponds to the maximum load timing in consideration of comparison of one of the optical images 80 with the optical images at previous and following timings, a position and an angle of the leg, and the like. As a result, the physician can identify the X-ray image 70 that corresponds to the maximum load timing from the plurality of X-ray images 70. In other words, the controller 50 executes control for identifiably displaying the X-ray image 70 that corresponds to the maximum load timing from the plurality of X-ray images 70 on the display by displaying the plurality of X-ray images 70 and the optical images 80 acquired (captured) in timings that are synchronized with detection of the X-rays using the X-ray detector 20 in alignment with each other on the display 60.

For example, the controller 50 displays each pair of the X-ray image 70 and the optical image 80 that are acquired at the same timing as each other in alignment with each other on right and left sides on the display 60 with the image pairs each of which includes the X-ray image 70 and the optical image 80 that are acquired at the same timing as each other being displayed in alignment with each other in a vertical direction in chronological order frame by frame. The X-ray image 70 and the optical image 80 displayed on the display 60 may have the same size as each other or different sizes from each other.

In the first embodiment, the physician who watches the display 60 can identify the optical image 80 that corresponds to the maximum load timing based on the plurality of optical images 80 displayed on the display 60. Also, the physician can identify the X-ray image 70 that is displayed in association with (in alignment with) the optical image 80 that corresponds to the maximum load timing as the X-ray image 70 that corresponds to the maximum load timing.

### X-Ray Image Display Method of First Embodiment

Control processing of an X-ray image display method using the X-ray imaging system 100 according to the first embodiment is now described with reference to FIG. 5. The control processing of the image display method including steps 401 to 406 is performed by the controller 50.

The frame rates of the X-ray images 70 and the optical images 80 are first set in step 401. In other words, the X-ray irradiation rate of the X-ray irradiator 10 and an acquisition rate of the detection signals of the X-rays detected by the X-ray detector 20 are set to synchronize with an acquisition rate of the optical images 80 captured by the optical image capturer 30.

Subsequently, in step 402, the subject 101 during the walking action as the loaded action is successively irradiated with the X-rays at the set irradiation rate. Subsequently, in step 403, the X-rays that pass through the subject 101 are detected by the X-ray detector 20. Subsequently, in step 404, the plurality of X-ray images 70 are generated and acquired by acquiring signals representing detection results of the X-rays detected.

Subsequently, in step 405, the optical images 80 are acquired as the load information to identify the state of the load applied to the leg of the subject 101 in the walking action separately from the X-ray detection. In the first embodiment, the optical images 80 as video are captured by the optical image capturer 30 at the frame rate that is set to synchronize with the timing of X-ray irradiation in step 402. The optical images 80 (the plurality of optical images 80 as still images) as video are generated based on the signals from the optical image capturer 30.

Subsequently, in step 406, the X-ray images 70 generated by the X-ray detection are displayed on the display 60. In the first embodiment, in step 406, the X-ray image 70 that corresponds to the maximum load timing in the walking action of the subject 101 is identifiably displayed on the display 60 based on the optical images 80 acquired. In other words, the X-ray image 70 that corresponds to the maximum load timing is identifiably displayed from the plurality of X-ray images 70 by displaying the plurality of X-ray images 70 and the optical images 80 acquired in timings that are synchronized with detection of the X-rays using the X-ray detector 20 in alignment with each other on the display 60.

Any one of the control process for generating the X-ray images 70 in step 404 and the control process for acquiring (generating) the optical images 80 in step 405 can be executed prior to another.

### Advantages of First Embodiment

In the first embodiment, the following advantages are obtained.

In the first embodiment, as described above, at least the X-ray image 70 that corresponds to a maximum load timing in which a largest load is applied to one of legs of the subject 101 in walking action (loaded action) based on the optical image 80 (load information) acquired is identifiably displayed. Accordingly, since the X-ray image 70 that corresponds to the maximum load timing is identifiably displayed, easy identification of the X-ray image 70 that corresponds to the maximum load timing in which the largest load is applied to the one of the legs can be realized by visual recognition of the X-ray image 70 displayed that corresponds to the maximum load timing.

Also, since the optical image capturer 30 (load information acquirer) acquires the optical images 80 (load information) to identify a state of the load applied to the leg in the loaded action of the subject 101 separately from the detection of the X-rays using the X-ray detector 20, it is possible to reduce a load of control processing on the controller 50 as compared with a case in which image processing for extracting bone parts from the X-ray images 70 is performed and processing for identifying the state of the load applied to the legs is performed based on positions and shapes of the extracted bone parts.

In addition, the following additional advantages can be obtained by the first embodiment added with configurations discussed below.

That is, in the first embodiment, as described above, the controller 50 controls the display 60 to identifiably display the X-ray image 70 which is captured by capturing an image of at least one of an ankle joint, a knee joint, and a hip joint in the one of the legs and that corresponds to the maximum load timing based on the optical image 80 (load information) acquired by the optical image capturer 30 (load information acquirer). According to this configuration, in a case in which the state of the joint under the loaded action is observed by confirming the X-ray images 70, which are captured by capturing an image of at least one of the ankle joint, the knee joint and the hip joint, it is possible to realize effective and easy identification of the X-ray image 70 that corresponds to the maximum load timing in which the largest load is applied to the one of the legs.

Also, in the first embodiment, the X-ray imaging system 100 includes the optical image capturer 30 for capturing the optical images 80 of the subject 101 during the loaded action. Here, since images of states of an exterior of the subject 101 are captured as the optical images 80 captured by the optical image capturer 30, easier identification of a state of the load applied to the leg in the loaded action can be realized. Accordingly, easier identification of the X-ray image 70 that corresponds to the maximum load timing can be realized by identification of the X-ray image 70 that corresponds to the maximum load timing based on the optical images 80.

Also, in the first embodiment, the X-ray imaging system 100 includes the optical image capturer 30 for capturing the optical images 80 of the subject 101 during the loaded action. The optical image capturer 30 is arranged in the collimator 12, which is at least one of the support 41 supporting the X-ray irradiator 10 and the collimator 12 for specifying an irradiation field of the X-rays with which the subject is irradiated by the X-ray irradiator 10. According to this configuration, since the optical image capturer 30 is arranged in the collimator 12, which is at least one of the support 41 supporting the X-ray irradiator 10 and the collimator 12, a direction in which the X-rays are emitted for irradiation by the X-ray irradiator 10 can agree with a direction in which the optical images 80 are captured by the optical image capturer 30. Consequently, further easier identification of the X-ray image 70 that corresponds to the maximum load timing can be realized by visual comparison of the optical images 80 with the X-ray images 70.

Also, in the first embodiment, the X-ray imaging system 100 includes the optical image capturer 30 for capturing the optical images 80 of the subject 101 during the loaded action. The controller 50 displays the X-ray image 70 that corresponds to the maximum load timing in association with the optical image 80 captured by the optical image capturer 30. According to this configuration, since the X-ray image 70 that corresponds to the maximum load timing is displayed in association with the optical image 80 captured, easy recognition whether the X-ray image 70 that is associated with the optical image 80 is the X-ray image 70 that corresponds to the maximum load timing can be realized by visual recognition of the optical image 80. Consequently, further easier identification of the X-ray image 70 that corresponds to the maximum load timing can be realized.

Also, in the first embodiment, the controller 50 displays the X-ray image 70 that corresponds to the maximum load timing and the optical image 80 that corresponds to the maximum load timing in alignment with each other on the display 60. According to this configuration, since the X-ray image 70 and the optical image 80 displayed in alignment with each other on the display 60 can be easily compared with each other, further easier identification of the X-ray image 70 that corresponds to the maximum load timing can be realized by visual recognition of the optical image 80 that corresponds to the maximum load timing.

Also, in the first embodiment, the controller 50 controls the display 60 to identifiably display the X-ray image 70 that corresponds to the maximum load timing from a plurality of X-ray images 70 acquired by successively irradiating the subject 101 who is in the loaded action with the X-rays. According to this configuration, the visually recognition of the plurality of X-ray images 70 acquired by successively irradiating the subject with X-rays allows confirmation of movements of the joint and bones of the subject 101, which change with the loaded action, and allows easy identification of the X-ray image 70 that corresponds to the maximum load timing in the loaded action.

Also, in the first embodiment, the controller controls 50 the display to identifiably display the X-ray image 70 that corresponds to the maximum load timing from the plurality of X-ray images 70 by displaying the plurality of X-ray images 70 that include the X-ray image 70 that corresponds to the maximum load timing and the optical images 80 acquired in timings that are synchronized with detection of the X-rays using the X-ray detector 20 in alignment with each other on the display 60. According to this configuration, since the plurality of X-ray images 70 acquired by successively irradiating the subject with X-rays and the optical images 80 acquired in timings that are synchronized with the plurality of X-ray images are displayed in alignment with each other, identification of the X-ray image 70 that corresponds to the maximum load timing from the plurality of X-ray images 70 can be easily made by determination of the captured optical images 80 of the exterior of the subject 101.

### Second Embodiment

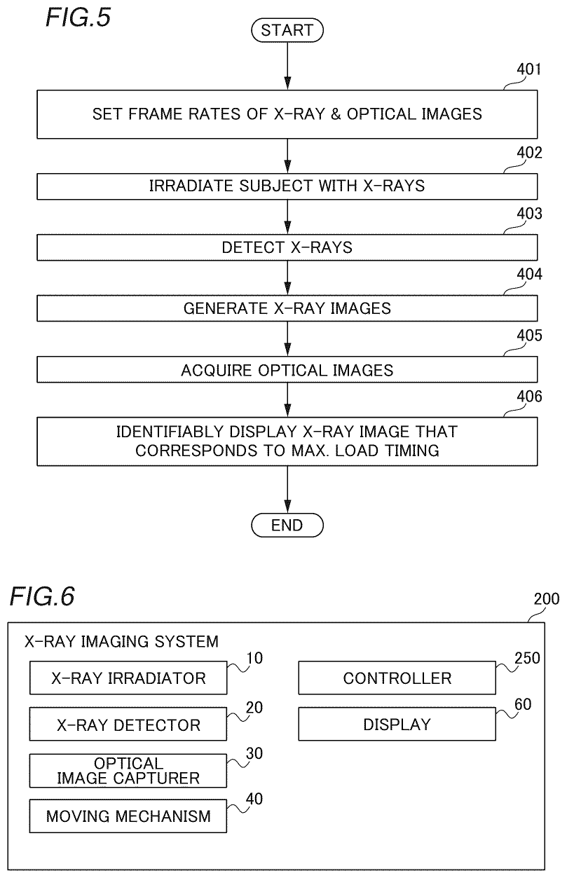

The following description describes a configuration of an X-ray imaging system 200 according to a second embodiment with reference to FIGS. 6 to 10. Dissimilar to the first embodiment in which a physician identifies the X-ray image 70 that corresponds to the maximum load timing based on the optical images 80 displayed, the X-ray image 70 that corresponds to the maximum load timing is automatically identified by control processing by using a controller 250 in this second embodiment. The same configurations in the Figures as those of the first embodiment are illustrated with the same reference numerals, and their description is omitted.

### Configuration of X-Ray Imaging System of Second Embodiment

As shown in FIG. 6, the X-ray imaging system 200 according to the second embodiment includes the X-ray irradiator 10, the X-ray detector 20, the optical image capturer 30, the moving mechanism 40, the controller 250, and the display 60. Similar to the controller 50 according to the first embodiment, the controller 250 performs control of X-ray imaging using the X-ray irradiator 10 and the X-ray detector 20, and control relating to capture of optical images 80 using the optical image capturer 30. Here, the controller 250 has a hardware configuration similar to the controller 50 according to the first embodiment.

In the second embodiment, the controller 250 acquires the maximum load timing based on the optical images 80 acquired by the optical image capturer 30. Specifically, the controller 250 acquires the maximum load timing on a basis of a position of another of the legs of the subject 101 in a height direction based on the optical images 80 acquired as the load information. The controller 250 controls the display 60 to identifiably display the X-ray image 70 that corresponds to the maximum load timing based on the maximum load timing acquired.

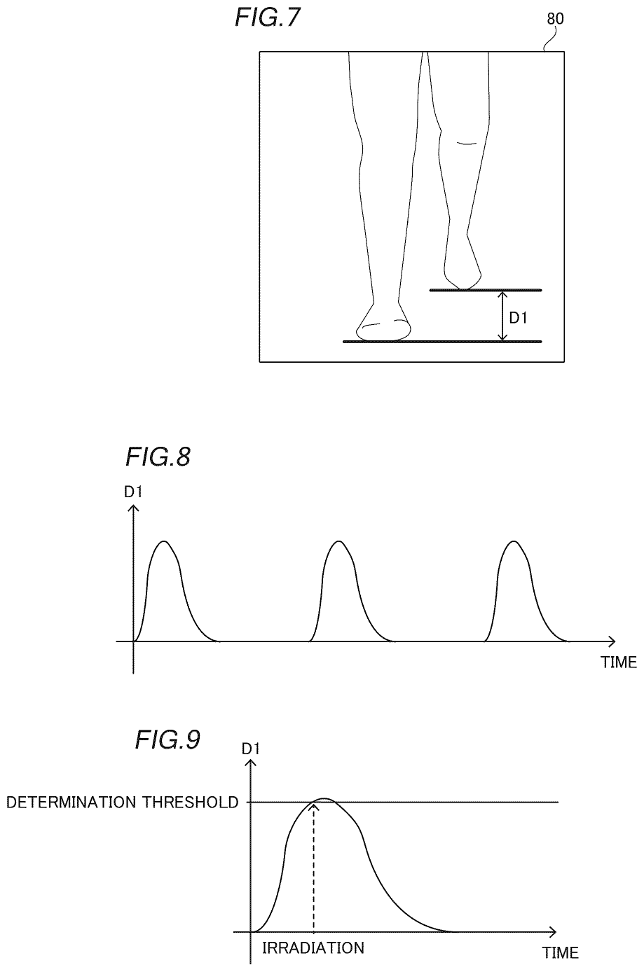

In the second embodiment, as shown in FIG. 7, prior to X-ray imaging, the controller 250 acquires the optical images 80 of the subject 101 in walking action. For example, prior to X-ray imaging, the controller 250 acquires the optical images 80 as video by capturing images of the subject 101 who repeats the walking action by using the optical image capturer 30. The controller 250 acquires the optical image 80, which are acquired as video, as a plurality of optical images 80 each of which is a still image for each frame, and also acquires the position of the another of the legs in the height direction in each of the plurality of optical images 80.

The controller 250 acquires the position of a left leg as the another of the legs in the height direction in a case in which X-ray images 70 of the knee joint of a right leg as the one of the legs are acquired. The controller 250 acquires the position of the left leg in a vertical direction as the height direction by applying image processing, such as pattern matching, to each of the plurality of optical images 80 acquired. The controller 250 detects, for example, a distance D1, which is a difference between the position of a toe of the right leg and a toe of the left leg in an upward/downward direction of each optical image 80, as the position of the left leg in the height direction.

As shown in FIG. 8, a value of the distance D1 periodically rises and drops in the walking action. A timing in which the distance D1 becomes the largest in one cycle of walking action is the maximum load timing in which the largest load is applied to the right leg as the one of the legs in the one cycle of walking action. The controller 250 acquires the optical images 80 as video for a plurality of cycles of walking action prior to X-ray imaging. The controller 250 acquires changes of the distance D1 in the walking action of the subject 101 for the plurality of cycles by applying processing for detecting the distance D1 to each of the plurality of optical images 80 acquired as still images for each frame of the video.

As shown in FIG. 9, in the second embodiment, the controller 250 is configured to acquire the maximum load timing based on the value of distance D1 and a determination threshold in the X-ray imaging, and to direct the X-ray irradiator 10 to irradiate the subject with X-rays in the maximum load timing acquired. The controller 250 sets the determination threshold, which is used to determine the maximum load timing, based on periodic values of the distance D1 in the walking action of the subject 101 acquired for the plurality of cycles prior to X-ray imaging. For example, the determination threshold can be acquired based on the maximum value of the distance D1 acquired in the plurality of cycles prior to X-ray imaging. A value that is obtained by multiplying an average of the maximum values of the distance D1 in each of the plurality of cycles by a predetermined ratio can be set as the determination threshold, for example. Here, the determination threshold may be a predetermined value that is previously set.

In the X-ray imaging, the controller 250 acquires the optical images 80 captured by the optical image capturer 30 as real-time video, and acquires a timing in which a magnitude of the distance D1 detected in the acquired optical image 80 exceeds the set determination threshold as the maximum load timing. The controller 250 is configured to direct the X-ray irradiator 10 to irradiate the subject with X-rays in a timing in which the value of the distance D1 detected in the optical image 80 acquired in real time during the loaded action exceeds the determination threshold in the X-ray imaging. Accordingly, in the second embodiment, the X-ray image 70 that corresponds to the maximum load timing is captured by controlling timing of X-ray irradiation based on the optical images 80.

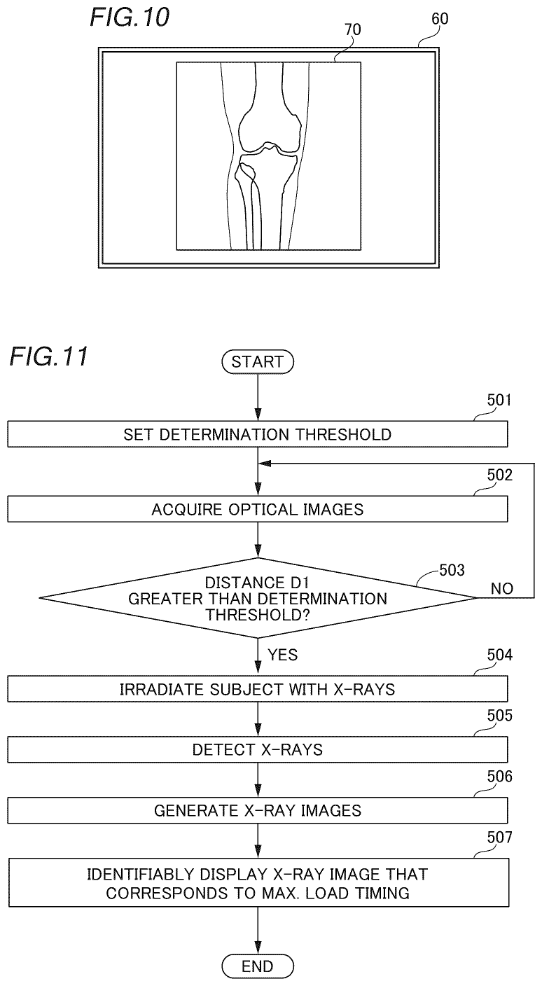

As shown in FIG. 10, the controller 250 displays one X-ray image 70 that is generated by detecting the X-rays with which the subject is irradiated in the maximum load timing on the display 60 as the X-ray image 70 that corresponds to the maximum load timing. That is, dissimilar to the first embodiment in which one X-ray image 70 that corresponds to the maximum load timing is identifiably displayed by displaying the plurality of X-ray images 70, the X-ray image 70 that corresponds to the maximum load timing is identifiably displayed by displaying only one X-ray image 70 that corresponds to the maximum load timing on the display 60 in the second embodiment. Other configurations of the second embodiment are the same as the first embodiment above.

### X-Ray Image Display Method of Second Embodiment

Control processing of an X-ray image display method using the X-ray imaging system 200 according to the second embodiment is now described with reference to FIG. 11. The control processing of the X-ray image display method including steps 501 to 507 is performed by the controller 250.

In step 501, the determination threshold is first set. For example, the distance D1, which is the difference between the position of the toe of the right leg and the position of the toe of the left leg in the upward/downward direction of the optical image 80, is acquired based on the optical image 80 acquired in the walking action for the plurality of cycles as a value representing the position of the another (left leg) of the legs of the subject 101 in the height direction. Subsequently, the determination threshold is set to determine the maximum load timing in one cycle based on periodic change of the value of the distance D1.

Subsequently, the optical image 80 is acquired for X-ray imaging in step 502. In order to irradiate the subject with X-rays in the maximum load timing, the optical images 80 of the subject 101 during the walking action are acquired as video. The optical images 80 are successively acquired in timings corresponding to a predetermined frame rate to form video with the predetermined frame rate.

In step 503, the distance D1 is acquired based on the optical image 80 acquired in step 502, and it is determined whether the acquired distance D1 is greater than the determination threshold, which is set in step 501. If it is determined that the distance D1 is greater than the determination threshold, the procedure goes to step 504. If it is determined that the distance D1 is not greater than the determination threshold, the procedure goes back to step 502 so that the optical image 80 of a new frame is acquired. Here, the control process in step 503 is executed every when the optical image 80, which is a still image as one frame of video, is acquired for each predetermined frame rate in step 502.

In step 504, the subject is irradiated with X-rays. In other words, it is determined that the maximum load timing is detected if the value of the detected distance D1 in the acquired optical image 80 is greater than the predetermined determination threshold. Correspondingly, the subject is irradiated with the X-rays in the maximum load timing.

Subsequently, in step 505, the X-rays with which the subject is irradiated in the maximum load timing are detected. Subsequently, in step 506, the X-ray image 70 that corresponds to the maximum load timing is generated based on the X-rays detected. Subsequently, in step 507, the X-ray image 70 that corresponds to the generated maximum load timing is identifiably displayed on the display 60. In step 507, the X-ray image 70 that corresponds to the maximum load timing is identifiably displayed on the display 60 by displaying only one X-ray image 70 that corresponds to the maximum load timing, for example.

### Advantages of Second Embodiment

In the second embodiment, the following advantages are obtained.

In the second embodiment, the controller 250 acquires the maximum load timing based on the optical images 80 (load information) acquired by the optical image capturer 30 (load information acquirer). Also, the controller 250 controls the display 60 to identifiably display the X-ray image 70 that corresponds to the maximum load timing based on the maximum load timing acquired. According to this configuration, since the maximum load timing is automatically acquired by the controller 250, the X-ray image 70 that corresponds to the maximum load timing can be automatically identified by control processing by using the controller 250. Consequently, it is possible to reduce time and effort to identify the X-ray image 70 that corresponds to the maximum load timing.

Also, in the second embodiment, the X-ray imaging system 200 includes the optical image capturer 30 for capturing the optical images 80 of the subject 101 during the loaded action. The controller acquires 250 the maximum load timing on a basis of a position of another of the legs of the subject 101 in a height direction, which is at least one of the position of the another of the legs of the subject 101 in the height direction and an inclination of a body axis of the subject 101, based on the optical images 80 acquired as the load information. According to this configuration, the maximum load timing can be easily automatically acquired by determining the positions of the another of the legs of the subject 101 in the height direction in the optical images 80. Consequently, it is possible to reduce a processing load on the control processing for acquiring the maximum load timing.

Also, in the second embodiment, the controller 250 is configured to direct the X-ray irradiator 10 to irradiate the subject with X-rays in the maximum load timing acquired. According to this configuration, it is possible to prevent a dose of the X-rays with which the subject 101 is irradiated from increasing as compared with a case in which the plurality of X-ray images 70 are acquired by successively irradiating the subject with the X-rays, while it is possible to easily acquire the X-ray image 70 that corresponds to the maximum load timing.

The other advantages of the second embodiment are similar to the first embodiment.

### Third Embodiment

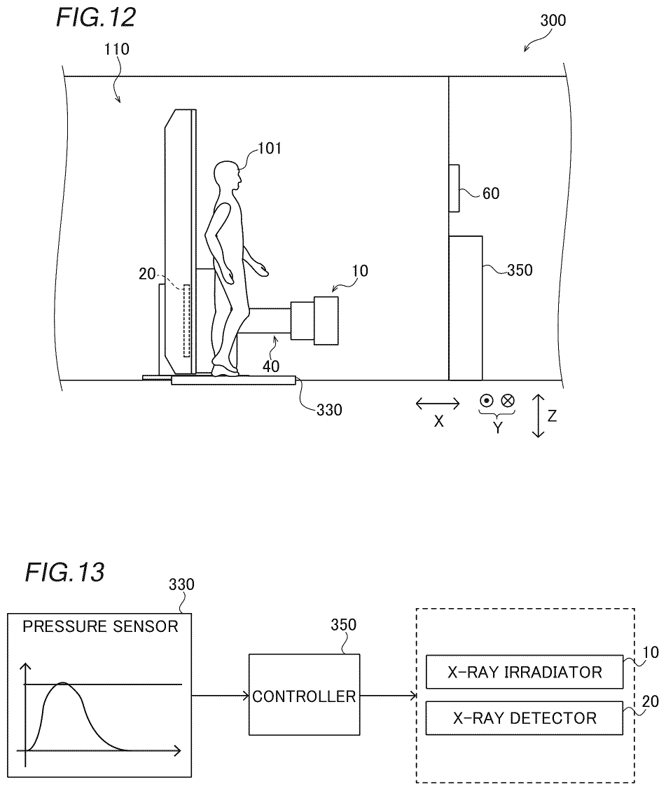

The following description describes a configuration of an X-ray imaging system 300 according to a third embodiment with reference to FIGS. 12 and 13. Dissimilar to the first and second embodiments in which the optical image capturer 30 for capturing the optical images 80 is included, a pressure sensor 330 is included in the third embodiment. The same configurations in the Figures as those of the first and second embodiments are illustrated with the same reference numerals, and their description is omitted. The pressure sensor 330 is an example of the “load information acquirer” in the claims.

### Configuration of X-Ray Imaging System of Third Embodiment

As shown in FIG. 12, the X-ray imaging system 300 according to the third embodiment includes the X-ray irradiator 10, the X-ray detector 20, the pressure sensor 330, the moving mechanism 40, the controller 350, and the display 60. Similar to the controller 50 according to the first embodiment and the controller 250 according to the second embodiment, the controller 350 performs control of X-ray imaging using the X-ray irradiator 10 and the X-ray detector 20. In the third embodiment, the controller 350 controls detection of pressure using the pressure sensor 330 and analysis processing based on detection results using the pressure sensor 330. The controller 350 has a hardware configuration similar to the first and second embodiments.

The pressure sensor 330 detects pressures applied by the leg of the subject 101 during the walking movement as loaded action. The pressure sensor 330 is a sheet-shaped sensor arranged on a floor of the imaging room 110 to be stepped on by the subject 101 who is in the walking action. The pressure sensor 330 includes, for example, a strain gauge or a capacitive sensor. The pressure sensor 330 detects the pressure applied through the feet by the legs of the subject 101 during the walking action when the subject 101 steps on the pressure sensor, and outputs a signal representing the detection result to the controller 350. That is, the pressure sensor 330 acquires the detection result of the pressure applied by the leg as load information to identify a state of the load applied to the leg in the waling action as loaded action of the subject 101 separately from the detection of the X-rays using the X-ray detector 20.

As shown in FIG. 13, in the third embodiment, the controller 350 acquires the detection result acquired as load information by the pressure sensor 330. Subsequently, the controller 350 acquires the maximum load timing by detecting a magnitude of the pressure that is applied by the one of the legs based on the acquired detection result. Also, the controller 350 is configured to direct the X-ray irradiator 10 to irradiate the subject with X-rays in the maximum load timing acquired.

Similar to the second embodiment, the controller 350 sets a determination threshold for detecting the maximum load timing prior to X-ray imaging. Subsequently, the controller 350 determines a timing in which the magnitude of the pressure that is applied by the one of the legs is greater than the set determination threshold based on the detection result acquired from the pressure sensor 330 as the maximum load timing, and acquires the X-ray image 70 that corresponds to the maximum load timing by irradiating the subject with X-ray in a timing in which determining the maximum load timing.

Specifically, similar to the second embodiment, the controller 350 acquires periodic values of the magnitude of the pressure that is applied by the one of the legs based on the detection result from the pressure sensor 330 for a plurality of cycles in the walking action prior to X-ray imaging. In the walking action, the magnitude of pressure applied by the one of the legs periodically varies, and becomes the maximum value in the maximum load timing. Similar to the distance D1 in the second embodiment, the controller 350 sets the determination threshold for determining the maximum load timing based on periodic variation of the magnitude of the pressure that is applied by the one of the legs acquired prior to X-ray imaging. Subsequently, similar to the second embodiment, in a case of X-ray imaging, the controller 350 successively acquires the detection results from the pressure sensor 330, and determines whether the magnitude of the pressure that is applied by the one of the legs is greater than the set determination threshold every when acquiring each detection result. The controller 350 determines that the maximum load timing is detected in a timing in which the magnitude of the pressure that is applied by the one of the legs is greater than the set determination threshold, and performs control processing for X-ray imaging using the X-ray irradiator 10 and the X-ray detector 20. Subsequently, similar to the second embodiment, the controller 350 controls the display 60 to display the X-ray image 70 that corresponds to the maximum load timing acquired based on the detection result of the pressure sensor 330. The other configuration of the third embodiment is similar to the second embodiment.

### Advantages of Third Embodiment

In the third embodiment, the following advantages are obtained.

In the third embodiment, the X-ray imaging system 300 includes the pressure sensor 330 for detecting a pressure applied by the leg of the subject 101 during loaded action. According to this configuration, it is possible to easily and directly identify a state of a load applied to the leg in the loaded action based on a detection result using the pressure sensor 330. Accordingly, it is possible to more easily identify the X-ray image 70 that corresponds to the maximum load timing in which the largest load is applied to the one of the legs based on the detection result using the pressure sensor 330.

Also, in the third embodiment, the X-ray imaging system 300 includes the pressure sensor 330 for detecting the pressure applied by the leg of the subject 101 during the loaded action. The controller 350 acquires the maximum load timing on a basis of a magnitude of the pressure that is applied by the one of the legs, which is at least one of the magnitude of the pressure that is applied by the one of the legs, and a comparison between the pressure that is applied by the one of the legs and the pressure that is applied by the another of the legs, based on the detection results using the pressure sensor 330 acquired as the load information. According to this configuration, the maximum load timing can be easily automatically acquired by determining the magnitude of the pressure that is applied by the one of the legs. Consequently, it is possible to reduce a processing load on the control processing for acquiring the maximum load timing.

The other advantages of the third embodiment are similar to the first and second embodiments.

### Modified Embodiments

Note that the embodiment disclosed this time must be considered as illustrative in all points and not restrictive. The scope of the present invention is not shown by the above description of the embodiments but by the scope of claims for patent, and all modifications (modified examples) within the meaning and scope equivalent to the scope of claims for patent are further included.

For example, while the example in which a subject 101 who is in walking action as loaded action in which a load is applied to legs of the subject is irradiated with X-rays has been shown in the aforementioned first to third embodiments, the present invention is not limited to this. In the present invention, the subject may exercise stepping up/down action in which he or she steps up and down stairs, or bending/stretching action in a standing posture.

While the example in which the X-ray image 70 which is captured by capturing an image of a knee joint and that corresponds to the maximum load timing is identifiably displayed has been shown in the aforementioned first to third embodiments, the present invention is not limited to this. In the present invention, an X-ray image which is captured by capturing an image of at least one of an ankle joint, the knee joint and a hip joint and that corresponds to the maximum load timing may be identifiably displayed on the display. Also, the X-ray image to be identifiably displayed is not limited to an X-ray image of the joint of the leg, but an X-ray image that includes a pelvis or spine and corresponds to the maximum load timing may be identifiably displayed.

While the example in which the X-ray images 70, which are captured by capturing an image of the knee joint, and the optical images 80 including parts from knee joints to toes are acquired has been shown in the aforementioned first and second embodiments, the present invention is not limited to this. In the present invention, the X-ray image and the optical image may have the same captured area as each other or different areas from each other. For example, in a case in which an X-ray image of the knee joint is captured, an optical image of the entire body of the subject may be acquired.

While the example in which the optical image capturer 30 is arranged to acquire the optical images 80 as load information to identify a state of the load applied to the leg has been shown in the aforementioned first and second embodiments, and the example in which the pressure sensor 330 is arranged to acquire a detection result of a pressure applied by the leg as load information to identify a state of the load applied to the leg has been shown in the aforementioned third embodiment, the present invention is not limited to this. In the present invention, another detection sensor for detecting action of the subject may be arranged as the load information acquirer. For example, a gyro sensor to be worn on the body of the subject may be included as the load information acquirer. Also, a non-contact capacitive sensor, magnetic sensor, or photoelectric sensor may be provided to identify the state of loaded action of the subject. Also, a plurality of types of sensors may also be combined to acquire the load information. For example, the load information may be acquired by combining the optical image capturer with the pressure sensor. That is, a plurality of types of load information may be displayed together with the X-ray image, or a single type of load information may be acquired by averaging results acquired based on the plurality of types of load information.

While the example in which the optical image capturer 30 is arranged in the collimator 12 for specifying an irradiation field of the X-ray irradiator 10 has been shown in the aforementioned first and second embodiments, the present invention is not limited to this. In the present invention, the optical image capturer may be arranged on the support supporting the X-ray irradiator. Also, the optical image capturer may be arranged at a position spaced away from the X-ray irradiator, such as on a wall or a ceiling surface of the imaging room. For example, the optical image capturer may be arranged on an X-ray detector side. Also, the optical image capturer may capture images of a part of the subject different from a part of the subject that is an imaging target of the X-ray imaging.

While the example in which the X-ray image 70 is displayed in association with the optical image 80 by displaying the X-ray image 70 in alignment with the optical image 80 has been shown in the aforementioned first embodiment, the present invention is not limited to this. In the present invention, the X-ray image that corresponds to an optical image selected may be displayed when an operation for selecting the optical image is accepted.

While the example in which the frame rate of the X-ray images 70 and the frame rate of the optical images 80 are synchronized with each other to bring agreement of the timings in which the X-ray images 70 as video are captured with the timings in which the optical images 80 as video are captured has been shown in the aforementioned first embodiment, the present invention is not limited to this. In the present invention, the frame rate of the X-ray images may be different from the frame rate of the optical images. Also, in a case in which the frame rate of the X-ray images agrees with the frame rate of the optical images, a timing in which each X-ray image is captured may disagree with a timing in which each optical image is captured. Similar to this, in a case in which the load information acquirer is another load information acquirer different from the optical image capturer, a rate of acquisition of load information may agree or disagree with the frame rate of X-ray images.

While the example in which the maximum load timing is acquired on a basis of a position of another of the legs of the subject 101 in a height direction has been shown in the aforementioned second embodiment, the present invention is not limited to this. In the present invention, the maximum load timing may be acquired on a basis of an inclination of a body axis of the subject. Also, in a case in which the position of another of the legs of the subject in the height direction is detected, the position of the another of the legs of the subject in the height may be detected by detecting a position of a floor surface in each optical image. Also, the maximum load timing in loaded action may be detected by detecting movement of a part other than the legs, such as swinging of arms based on each optical image.

While the example in which the maximum load timing is acquired by detecting a magnitude of a pressure applied by the one of the legs based on detection results acquired as load information by the pressure sensor 330 has been shown in the aforementioned third embodiment, the present invention is not limited to this. In the present invention, the maximum load timing may be acquired on a basis of a comparison between the pressure that is applied by the one of the legs and the pressure that is applied by the another of the legs based on detection results using the pressure sensor acquired as the load information. For example, a difference or a ratio between the pressure applied by the one of the legs and the pressure applied by the another of the legs may be acquired. Also, the maximum load timing may be acquired by combining the magnitude of the pressure that is applied by the one of the legs with the comparison between the pressure that is applied by the one of the legs and the pressure that is applied by the another of the legs. The maximum load timing of the one of the legs may be acquired based on the pressure applied by the another of the legs.

While the example in which the maximum load timing is acquired, and one X-ray image 70 that corresponds to the maximum load timing is displayed by irradiating the subject with X-ray by the X-ray irradiator 10 in the maximum load timing acquired has been shown in the aforementioned second and third embodiments, the present invention is not limited to this. In the present invention, alternatively, a plurality of X-ray images may be generated and displayed with the X-ray image that corresponds to the maximum load timing being identifiably displayed. For example, when the plurality of X-ray images is displayed in alignment with each other, the X-ray image that is determined as one corresponding to the maximum load timing may be identifiably displayed by highlighting the X-ray images determined, such as by enclosing the X-ray images determined with a frame line. In this alternative case, if the maximum load timing acquired disagrees with each of timings at which the plurality of X-ray images are captured, the X-ray image that is captured in a timing that is closer to the maximum load timing may be acquired as the X-ray image that corresponds to the maximum load timing. Also, the plurality of X-ray images and a plurality items of load information (a plurality of optical images or detection results of the pressure sensor) may be displayed, and the X-ray image that corresponds to the maximum load timing may be identifiably displayed by highlighting the item of load information that corresponds to the maximum load timing in the plurality items of load information. Also, in a case in which the maximum load timing is not acquired, instead of the optical images, numerical values indicating the detection results (load information) acquired by the load information acquirer other than the optical image capturer, such as the pressure sensor, may be displayed in association with (in alignment with) the X-ray images. Also, in a case in which the optical image capturer is arranged as the load information acquirer, instead of the optical images, detection values such as positions of the leg acquired based on the optical images may be displayed in association with the X-ray images.

While the example in which the maximum load timing is determined based on a set determination threshold has been shown in the aforementioned second and third embodiments, the present invention is not limited to this. In the present invention, the maximum load timing may be acquired without setting the determination threshold. For example, the maximum load timing may be predicted and acquired by acquiring load information, such as optical images, from a subject who repeatedly exercises loaded action in predetermined cycles, and by acquiring the cycle to be taken for a single complete loaded action.

While the example in which the controllers 50, 250, and 350 control X-ray imaging, generation of X-ray images, capture of optical images and acquisition of pressure sensor detection results (acquisition of load information), and acquisition of the maximum load timing based on the load information has been shown in the aforementioned first to third embodiments, the present invention is not limited to this. In the present invention, one or some of the X-ray imaging, the generation of X-ray images, the acquisition of load information, and the acquisition of the maximum load timing may be controlled by control apparatuses different from each other or by a combination of a plurality of control apparatuses. Also, the control apparatus that makes up the controller may include one or some of a personal computer, a processor, a circuit and an integrated circuit.

While the example in which X-ray imaging of the subject 101 who is in the loaded action is performed by the X-ray fluoroscopy system for rotating the X-ray irradiator 10 together with the bed 21 in which the X-ray detector 20 is arranged has been shown in the aforementioned first to third embodiments, the present invention is not limited to this. In the present invention, the X-ray irradiator and the X-ray detector may be spaced away from each other. For example, X-ray imaging of the subject who is in the loaded action may be performed by a typical ceiling-suspended type X-ray imaging apparatus including an X-ray irradiator supported by a support arranged on a ceiling. Also, X-ray imaging of the subject who is in the loaded action may be performed by a movable X-ray imaging apparatus for hospital rounds or an X-ray fluoroscopy including a C-arm.

### [Modes]

It is understood by those skilled in the art that the exemplary embodiments described above are specific examples of the following aspects.

### (Mode Item 1)

An X-ray imaging system according to mode item 1 includes an X-ray irradiator for irradiating a subject who is in loaded action in which a load is applied to legs of the subject with X-rays; an X-ray detector for detecting the X-rays with which the subject is irradiated by the X-ray irradiator and that pass through the subject; a load information acquirer for acquiring load information to identify a state of the load applied to the leg or the legs in the loaded action of the subject separately from the detection of the X-rays using the X-ray detector; a display for displaying an X-ray image(s) generated based on the detection of the X-rays using the X-ray detector; and a controller for executing control for identifiably displaying at least the X-ray image that corresponds to a maximum load timing in which a largest load is applied to one of the legs in the loaded action of the subject on the display based on the load information acquired by the load information acquirer.

### (Mode Item 2)

In the X-ray imaging system according to mode item 1, the controller executes control for identifiably displaying the X-ray image which is captured by capturing an image of at least one of an ankle joint, a knee joint and a hip joint in the one of the legs and that corresponds to the maximum load timing on the display based on the load information acquired by the load information acquirer.

### (Mode Item 3)

In the X-ray imaging system according to mode item 1 or 2, the load information acquirer includes at least one of an optical image capturer for capturing optical images of the subject during the loaded action and a pressure sensor for detecting pressures applied by the leg or the legs of the subject during the loaded action.

### (Mode Item 4)

In the X-ray imaging system according to mode item 3, the load information acquirer includes the optical image capturer for capturing the optical images of the subject during the loaded action; and the optical image capturer is arranged in at least one of a support supporting the X-ray irradiator and a collimator for specifying an irradiation field of the X-rays with which the subject is irradiated by the X-ray irradiator.

### (Mode Item 5)

In the X-ray imaging system according to mode item 3 or 4, the load information acquirer includes the optical image capturer for capturing the optical images of the subject during the loaded action; and the controller displays the X-ray image that corresponds to the maximum load timing in association with the optical image captured by the optical image capturer.

### (Mode Item 6)

In the X-ray imaging system according to mode item 5, the controller displays the X-ray image that corresponds to the maximum load timing and the optical image that corresponds to the maximum load timing in alignment with each other on the display.

### (Mode Item 7)

In the X-ray imaging system according to any of mode items 1 to 6, the controller executes control for identifiably displaying the X-ray image that corresponds to the maximum load timing on the display from a plurality of X-ray images acquired by successively irradiating the subject who is in the loaded action with the X-rays.

### (Mode Item 8)

In the X-ray imaging system according to mode item 7, the controller executes control for identifiably displaying the X-ray image that corresponds to the maximum load timing on the display from the plurality of X-ray images by displaying the plurality of X-ray images that include the X-ray image that corresponds to the maximum load timing and the load information acquired in a timing that is synchronized with detection of the X-rays using the X-ray detector in alignment with each other on the display.

### (Mode Item 9)

In the X-ray imaging system according to any of mode items 1 to 8, the controller executes control for acquiring the maximum load timing based on the load information acquired by the load information acquirer, and control for identifiably displaying the X-ray image that corresponds to the maximum load timing based on the maximum load timing acquired on the display.

### (Mode Item 10)

In the X-ray imaging system according to mode item 9, the load information acquirer includes an optical image capturer for capturing optical images of the subject during the loaded action; and the controller acquires the maximum load timing on a basis of at least one of a position of another of the legs of the subject in a height direction and an inclination of a body axis of the subject based on the optical images acquired as the load information.

### (Mode Item 11)

In the X-ray imaging system according to mode item 9, the load information acquirer includes a pressure sensor for detecting a pressure applied by the leg or the legs of the subject during the loaded action; and the controller acquires the maximum load timing on a basis of at least one of a magnitude of the pressure that is applied by the one of the legs, and a comparison between the pressure that is applied by the one of the legs and the pressure that is applied by another of the legs based on detection results using the pressure sensor acquired as the load information.

### (Mode Item 12)

In the X-ray imaging system according to any of mode items 9 to 11, the controller is configured to direct the X-ray irradiator to irradiate the subject with the X-rays in the maximum load timing acquired.

### (Mode Item 13)

An X-ray image display method according to mode item 13 includes a step of irradiating a subject who is in loaded action in which a load is applied to legs of the subject with X-rays; a step of detecting the X-rays that pass through the subject; a step of acquiring load information to identify a state of the load applied to the leg or the legs in the loaded action of the subject separately from the detection of the X-rays; and a step of displaying an X-ray image(s) generated based on the detection of the X-rays, wherein the step of displaying the X-ray image(s) includes a step of identifiably displaying at least the X-ray image that corresponds to a maximum load timing in which a largest load is applied to one of the legs in the loaded action of the subject based on the load information acquired.

What is claimed is:

1. An X-ray imaging system comprising:
an X-ray irradiator for irradiating a subject who is in loaded action in which a load is applied to legs of the subject with X-rays;
an X-ray detector for detecting the X-rays with which the subject is irradiated by the X-ray irradiator and that pass through the subject;
a load information acquirer for acquiring load information to identify a state of the load applied to the leg or the legs in the loaded action of the subject separately from the detection of the X-rays using the X-ray detector;
a display for displaying an X-ray image(s) generated based on the detection of the X-rays using the X-ray detector; and
a controller for executing control for identifiably displaying at least the X-ray image that corresponds to a maximum load timing in which a largest load is applied to one of the legs in the loaded action of the subject on the display based on the load information acquired by the load information acquirer.

2. The X-ray imaging system according to claim 1, wherein the controller executes control for identifiably displaying the X-ray image which is captured by capturing an image of at least one of an ankle joint, a knee joint, and a hip joint in the one of the legs and that corresponds to the maximum load timing on the display based on the load information acquired by the load information acquirer.

3. The X-ray imaging system according to claim 1, wherein the load information acquirer includes at least one of an optical image capturer for capturing optical images of the subject during the loaded action and a pressure sensor for detecting pressures applied by the leg or the legs of the subject during the loaded action.

4. The X-ray imaging system according to claim 3, wherein
the load information acquirer includes the optical image capturer for capturing the optical images of the subject during the loaded action; and
the optical image capturer is arranged in at least one of a support supporting the X-ray irradiator and a collimator for specifying an irradiation field of the X-rays with which the subject is irradiated by the X-ray irradiator.

5. The X-ray imaging system according to claim 3, wherein
the load information acquirer includes the optical image capturer for capturing the optical images of the subject during the loaded action; and
the controller displays the X-ray image that corresponds to the maximum load timing in association with the optical image captured by the optical image capturer.

6. The X-ray imaging system according to claim 5, wherein the controller displays the X-ray image that corresponds to the maximum load timing and the optical image that corresponds to the maximum load timing in alignment with each other on the display.

7. The X-ray imaging system according to claim 1, wherein the controller executes control for identifiably displaying the X-ray image that corresponds to the maximum load timing on the display from a plurality of X-ray images acquired by successively irradiating the subject who is in the loaded action with the X-rays.

8. The X-ray imaging system according to claim 7, wherein the controller executes control for identifiably displaying the X-ray image that corresponds to the maximum load timing on the display from the plurality of X-ray images by displaying the plurality of X-ray images that include the X-ray image that corresponds to the maximum load timing and the load information acquired in a timing that is synchronized with detection of the X-rays using the X-ray detector in alignment with each other on the display.

9. The X-ray imaging system according to claim 1, wherein the controller executes
control for acquiring the maximum load timing based on the load information acquired by the load information acquirer, and
control for identifiably displaying the X-ray image that corresponds to the maximum load timing based on the maximum load timing acquired on the display.

10. The X-ray imaging system according to claim 9, wherein
the load information acquirer includes an optical image capturer for capturing optical images of the subject during the loaded action; and
the controller acquires the maximum load timing on a basis of at least one of a position of another of the legs of the subject in a height direction and an inclination of a body axis of the subject based on the optical images acquired as the load information.

11. The X-ray imaging system according to claim 9, wherein
the load information acquirer includes a pressure sensor for detecting a pressure applied by the leg or the legs of the subject during the loaded action; and
the controller acquires the maximum load timing on a basis of at least one of a magnitude of the pressure that is applied by the one of the legs, and a comparison between the pressure that is applied by the one of the legs and the pressure that is applied by another of the legs based on detection results using the pressure sensor acquired as the load information.

12. The X-ray imaging system according to claim 9, wherein the controller is configured to direct the X-ray irradiator to irradiate the subject with the X-rays in the maximum load timing acquired.

13. An X-ray image display method comprising:
a step of irradiating a subject who is in loaded action in which a load is applied to legs of the subject with X-rays;
a step of detecting the X-rays that pass through the subject;
a step of acquiring load information to identify a state of the load applied to the leg or the legs in the loaded action of the subject separately from the detection of the X-rays; and
a step of displaying an X-ray image(s) generated based on the detection of the X-rays, wherein
the step of displaying the X-ray image(s) includes a step of identifiably displaying at least the X-ray image that corresponds to a maximum load timing in which a largest load is applied to one of the legs in the loaded action of the subject based on the load information acquired.
